SEQUENCE LISTING (30 sequences)

SEQ ID NO: 1 - RNA, 422 nt, Homo sapiens
  source 1..422: mol_type = mRNA
gaggccctgcccaatgagactctagggtccagtggatgccacagcccagcttggccctttccttccagatcctgggtactgaaagccttagggaagctggcctgagaggggaagcggccctaagggagtgtctaagaacaaaagcgacccattcagagactgtccctgaaacctagtactgccccccatgaggaaggaacagcaatggtgtcagtatccaggctttgtacagagtgcttttctgtttagtttttactttttttgttttgtttttttaaagatgaaataaagacccagggggagaatgggtgttgtatggggaggcaagtgtggggggtccttctccacacccactttgtccatttgcaaatatattttggaaaacagctaggcaccggcctatgtctgggggtggctctcat

SEQ ID NO: 2 - RNA, 424 nt, Homo sapiens
  source 1..424: mol_type = mRNA
atctgaggccctgcccaatgagactctagggtccagtggatgccacagcccagcttggccctttccttccagatcctgggtactgaaagccttagggaagctggcctgagaggggaagcggccctaagggagtgtctaagaacaaaagcgacccattcagagactgtccctgaaacctagtactgccccccatgaggaaggaacagcaatggtgtcagtatccaggctttgtacagagtgcttttctgtttagtttttactttttttgttttgtttttttaaagatgaaataaagacccagggggagaatgggtgttgtatggggaggcaagtgtggggggtccttctccacacccactttgtccatttgcaaatatattttggaaaacagctaggcaccggcctatgtctgggggtggctctc

SEQ ID NO: 3 - RNA, 424 nt, Homo sapiens
  source 1..424: mol_type = mRNA
gactgaggccctgcccaatgagactctagggtccagtggatgccacagcccagcttggccctttccttccagatcctgggtactgaaagccttagggaagctggcctgagaggggaagcggccctaagggagtgtctaagaacaaaagcgacccattcagagactgtccctgaaacctagtactgccccccatgaggaaggaacagcaatggtgtcagtatccaggctttgtacagagtgcttttctgtttagtttttactttttttgttttgtttttttaaagatgaaataaagacccagggggagaatgggtgttgtatggggaggcaagtgtggggggtccttctccacacccactttgtccatttgcaaatatattttggaaaacagctaggcaccggcctatgtctgggggtggctctt

SEQ ID NO: 4 - RNA, 337 nt, Homo sapiens
  source 1..337: mol_type = mRNA
atgtaactgcctcaaattggactttgggcataaaagaacttttttatgcttaccatcttttttttttctttaacagatttgtatttaaaaattgtttttaaaaaattttaagatttacacaatgtttctctgtaaatattgccattaaatgtaaataactttaataaaacgtttatagcagttacccagaatttcaatccaaaagtgggtgggggggggagggaggggagaaggcacctggccggggaactcagccaaggttgtgagggttgattattataacgacgaccctacaggtcggaaagcaggggaggatgcatttgaatccccgaagcaa

SEQ ID NO: 5 - RNA, 205 nt, Homo sapiens
  source 1..205: mol_type = mRNA
aaatggtaaactgcctcaaattggactttgggcataaaagaacttttttatgcttaccatcttttttttttctttaacagatttgtatttaagaattgtttttaaaaaattttaagatttacacaatgtttctctgtaaatattgccattaaatgtaaataactttaataaaacgtttatagcagttacacagaatttcaatccc

SEQ ID NO: 6 - RNA, 193 nt, Homo sapiens
  source 1..193: mol_type = mRNA
ctgcctcattggactttgggcataaaagaacttttttatgcttaccatcttttttttttctttaacagatttgtatttaagaattgtttttaaaaaattttaagatttacacaatgtttctctgtaaatattgccattaaatgtaaataactttaataaaacgtttatagcagttacccagaatttcaatcca

SEQ ID NO: 7 - RNA, 331 nt, Homo sapiens
  source 1..331: mol_type = mRNA
cgtggctggacctctgggcgtgaacgaacttttttatgcttaccatcttttttttttctttaacagatttgtatttaagaattgtttttaaaaaattttaagatttacacaaggtttctctgtaaatatggccattagggtaaataactttaataaaacgtttatggcagttacacggaatttcaatccacaatctgtccgtgagcccggcaatgatcgctaaaggccttctaccgacagatatagtgtctttcggcgagtagttgattattataacgacgaccctacaggtcggaaagcaggggaggaagcatttgaagcaagaaagcaa

SEQ ID NO: 8 - RNA, 381 nt, Homo sapiens
  source 1..381: mol_type = mRNA
tctattggattgtgggaataaaagaacttttttatgattaccatcttttttttttctttaacagatttgtatttaagaattgtttttaaaaaattttaagatttacacaatgtttctctgtaaatattgccattaaatgtaaataactttaataaaacgtttatagcagttacacagaatttcaatcccaatctctgcaacccaacacttgttccctatgttctcgatcccccaaaaagactcctgtgctacaagatatcaatcattccctaatttttttaacttaggcgcactcgcattcttccttttcacatcccaaaccccccctcccgttttgcaacccatgtccactcacctctctcctaccccatagaaactctg

SEQ ID NO: 9 - RNA, 192 nt, Homo sapiens
  source 1..192: mol_type = mRNA
gcctcattggactttgggcataaaagaacttttttatgcttaccatcttttttttttctttaacagatttgtatttaagaattgtttttaaaaaattttaaaatttacacaatgtttctctgtaaatattgccattaaaggtaaataactttaataaaacgtttatagcagttacccagaatttcaatccaa

SEQ ID NO: 10 - RNA, 590 nt, Homo sapiens
  source 1..590: mol_type = mRNA
aatgtagcctcattggactgtgggcataaaagaacttttttatgcttaccatctttttttttttttaacaattggatttaaaaattggttttaaaaaattttaagaattacaccaaggttctccggaaatattgccctttaatggtaataaattttataaaacgttttttctccttaccccgaattttcatccaatatgtctttttcatatactccaatgaatgtaacacaatgtatcactgggctttggccatacactataaactttcttaccgaatcccttgcttcccaatcataaaaaattcaccaattgctataattattgcaattgaaaaattcattgattctcaatcttgaccccccctcgcccccctggtatccccagaagagcccggttgaaattttttaaattcccaacccaatcccttttcttgctaggtgcctggaatgtgggggttttggaagtcttttaattgatccgcctcccctctaggacccctggcccttttgccaccaattcggtgtgaaaattccctacgccttgggccggacaacctacataccgtttaccctttttttacttgacaaat

SEQ ID NO: 11 - RNA, 620 nt, Homo sapiens
  source 1..620: mol_type = mRNA
ggatcaattggactttgggcataaaagaacttttttatgcttaccatcttttttttttctttaacagatttgtatttaagaattgtttttaaaaaattttaagatttacccaatgtttctctgtaaatattgccattaaatgtaaataactttaataaaacgtttatagcagttacacagaatttcaatccaatgtcgtttcatatactccaatgaatgtaacacaatgtatccctggtcaaacaaccttgaaccaaaactttccaaaccgaatctctgcttccccaacctttaaaaaacccacgaaaggctataataaatgtgatttgaaaaatcccatcaatcacgaacctttgaccgaccctcgcgccccttgttttccttaggcccccccgtttaaagtcctttaaatttccccccccaccacttttccttgttgagcctggcaattgggggttttgccagttcttttatttatccttccccccctatcgaccttcgggccccttttcaaccaaattggtgtgaaactccctccccccaagaccaagacccaacatcaccttgtcccacttgcttaataaccaaataaacctcattctttccaaattctattcccc

SEQ ID NO: 12 - RNA, 504 nt, synthetic construct
  misc_feature 1..504: note = Synthetic sequence based on MYC wherein stabilizing motifs have been selectively replaced by destabilizing motifs
  source 1..504: mol_type = other RNA
gagcaatcacctatgaacttgctgctaagttatgatcaaatgcaacctcacaaccttggctgagtcttgagactgaaagatttagccataatgtaaactgcctcaaattggactttgggcataaaagaaccctcctgcatgcttaccatccctcctgccctcctttaacagcctcgtaacttataattgacctcaaaaaacctcaagatttacacaatgtttctctgtaaatattgccattaaatgtaaataactttaataaaacgtttatagcagttacacagaatttcaatcctagtatatagtacctagtattataggtactataaaccctaacctcctgcatttaagtacattttgcactgcaaagtaagttatcctctattgacctcagaaaaaataaaataactggcaaatatatcattgagccaaatcttaagttgtgaatgctgcctctgctaacttatccctcccaaccaccaccatccctgctgccctcatcaa

SEQ ID NO: 13 - RNA, 501 nt, synthetic construct
  misc_feature 1..501: note = Synthetic sequence based on ERBB2 wherein stabilizing motifs have been selectively replaced by destabilizing motifs
  source 1..501: mol_type = other RNA
caggggaacctgccatgccaggaacctgtcctaaggaacctgccttcctgcttgagttcccagatggctggaaggggtccagcctcgttggaagaggaacagcactggggagtcttcgtggattctgaggccctgcccaatgagactctagggtccagtggatgccacagcccagcttggccctctccttccagatcctgggtactgaaagccttagggaagctggcctgagaggggaagcggccctaagggagtgtctaagaacaaaagcgacccattcagagactgtccctgaaacctagtactgccccccatgaggaaggaacagcaatggtgtcagtatccaggctgcgtacagagtgccctcctgcttagctgctaagttatctgcctcgctgcctcaaagatgaaataaagacccagggggagaatgggtgttgtatggggaggcaagtgtggggggtccttctccacacccactttgtccatttgcaaatatat

SEQ ID NO: 14 - RNA, 68 nt, Homo sapiens
  source 1..68: mol_type = mRNA
caggctttgtacagagtgcttttctgtttagtttttactttttttgttttgtttttttaaagatgaaa

SEQ ID NO: 15 - RNA, 71 nt, synthetic construct
  misc_feature 1..71: note = Synthetic sequence wherein stabilizing motifis have been selectively replaced by destabilizing motifs
  source 1..71: mol_type = other RNA
caggctgcgtacagagtgccctcctgcttagctgttaagttatctgcctcgctgcctcaaagatgaaataa

SEQ ID NO: 16 - DNA, 768 bp, synthetic construct
  misc_feature 1..768: note = Synthetic sequence based on ERBB2 wherein stabilizing motifs have been selectively replaced by destabilizing motifs
  source 1..768: mol_type = other DNA
gcccgcgaggacccgcccgagcttcgaaaaaaaaaagccatcgaggcaaaattcccccatctgcgtcagtcccctcaacttccgcctctacgcgggcctcgcggcagggcggtacgtcagccattctggtccgccgcgcgcacgctccgggcgccgggttccggcgttgtcagggtccgcggccctacgatgtgggcggtgtccaaggctgcgtagtggagcttgcaggctggagctcaggggaacctgccatgccaggaacctgtcctaaggaacctgccttcctgcttgagttcccagatggctggaaggggtccagcctcgttggaagaggaacagcactggggagtcttcgtggattctgaggccctgcccaatgagactctagggtccagtggatgccacagcccagcttggccctctccttccagatcctgggtactgaaagccttagggaagctggcctgagaggggaagcggccctaagggagtgtctaagaacaaaagcgacccattcagagactgtccctgaaacctagtactgccccccatgaggaaggaacagcaatggtgtcagtatccaggtcgcgttcagagtgccctcctgcttagctgctaagttatctgcctcgctgcctcaaagatgaaataaagacccagggggagaatgggtgttgtatggggaggcaagtgtggggggtccttctccacacccactttgtccatttgcaaatatataaaaaaaaaacctaggggccggccccgccg

SEQ ID NO: 17 - DNA, 690 bp, synthetic construct
  misc_feature 1..690: note = Synthetic sequence based on MYC wherein stabilizing motifs have been selectively replaced by destabilizing motifs
  source 1..690: mol_type = other DNA
ggacccgcccgagcttcgaaaaaaaaaagccatcgaggcaaaattcccccatctgcgtcagtcccctcaacttccgcctctacgcgggcctcgcggcagggcggtacgtcagccattctggtccgccgcgcgcatgcgtagtggagcttgcaggctggagctgagcaatcacctatgaacttgctgctaagttatgatcaaatgcaacctcacaaccttggctgagtcttgagactgaaagatttagccataatgtaaactgcctcaaattggactttgggcataaaagaaccctcctgcatgcttaccatccctcctgccctcctttaacagcctcgtaacttataattgacctcaaaaaacctcaagatttacacaatgtttctctgtaaatattgccattaaatgtaaataactttaataaaacgtttatagcagttacacagaatttcaatcctagtatatagtacctagtattataggtactataaaccctaacctcctgcatttaagtacattttgcactgcaaagtaagttatcctctattgacctcagaaaaaataaaataactggcaaatatatcattgagccaaatcttaagttgtgaatgctgcctctgctaacttatccctcccaaccaccaccatccctgctgccctcatcaaaaaacctaggggccggccccgccg

SEQ ID NO: 18 - DNA, 501 bp, synthetic construct
  misc_feature 1..501: note = Synthetic sequence based on ERBB2 wherein stabilizing motifs have been selectively replaced by destabilizing motifs
  source 1..501: mol_type = other DNA
caggggaacctgccatgccaggaacctgtcctaaggaacctgccttcctgcttgagttcccagatggctggaaggggtccagcctcgttggaagaggaacagcactggggagtcttcgtggattctgaggccctgcccaatgagactctagggtccagtggatgccacagcccagcttggccctctccttccagatcctgggtactgaaagccttagggaagctggcctgagaggggaagcggccctaagggagtgtctaagaacaaaagcgacccattcagagactgtccctgaaacctagtactgccccccatgaggaaggaacagcaatggtgtcagtatccaggtcgcgttcagagtgccctcctgcttagctgctaagttatctgcctcgctgcctcaaagatgaaataaagacccagggggagaatgggtgttgtatggggaggcaagtgtggggggtccttctccacacccactttgtccatttgcaaatatat

SEQ ID NO: 19 - DNA, 502 bp, synthetic construct
  misc_feature 1..502: note = Synthetic sequence based on MYC wherein stabilizing motifs have been selectively replaced by destabilizing motifs
  source 1..502: mol_type = other DNA
gagcaatcacctatgaacttgctgctaagttatgatcaaatgcaacctcacaaccttggctgagtcttgagactgaaagatttagccataatgtaaactgcctcaaattggactttgggcataaaagaaccctcctgcatgcttaccatccctcctgccctcctttaacagcctcgtaacttataattgacctcaaaaaacctcaagatttacacaatgtttctctgtaaatattgccattaaatgtaaataactttaataaaacgtttatagcagttacacagaatttcaatcctagtatatagtacctagtattataggtactataaaccctaacctcctgcatttaagtacattttgcactgcaaagtaagttatcctctattgacctcagaaaaaataaaataactggcaaatatatcattgagccaaatcttaagttgtgaatgctgcctctgctaacttatccctcccaaccaccaccatccctgctgccctcatc

SEQ ID NO: 20 - DNA, 20 bp, synthetic construct
  misc_feature 1..20: note = ERBB2 forward primer for use in PCR
  source 1..20: mol_type = other DNA
gcctcgttggaagaggaaca

SEQ ID NO: 21 - DNA, 20 bp, synthetic construct
  misc_feature 1..20: note = ERBB2 reverse primer for use in PCR
  source 1..20: mol_type = other DNA
agagccacccccagacatag

SEQ ID NO: 22 - DNA, 20 bp, synthetic construct
  misc_feature 1..20: note = MYC forward primer for use in PCR
  source 1..20: mol_type = other DNA
cctcacaaccttggctgagt

SEQ ID NO: 23 - DNA, 23 bp, synthetic construct
  misc_feature 1..23: note = MYC reverse primer for use in PCR
  source 1..23: mol_type = other DNA
ggattgaaattctgtgtaactgc

SEQ ID NO: 24 - DNA, 20 bp, synthetic construct
  misc_feature 1..20: note = DCP1A forward primer for use in PCR
  source 1..20: mol_type = other DNA
ccctcaacttccgcctctac

SEQ ID NO: 25 - DNA, 20 bp, synthetic construct
  misc_feature 1..20: note = DCP1A reverse primer for use in PCR
  source 1..20: mol_type = other DNA
cagcctgcaagctccactac

SEQ ID NO: 26 - RNA, 14 nt, synthetic construct
  misc_feature 1..14: note = digestion enhancing sequence
  source 1..14: mol_type = other RNA
ggacccgcccgagc

SEQ ID NO: 27 - RNA, 15 nt, synthetic construct
  misc_feature 1..15: note = digestion enhancing sequence
  source 1..15: mol_type = other RNA
gggccggccccgccg

SEQ ID NO: 28 - RNA, 22 nt, synthetic construct
  misc_feature 1..22: note = Digestion enhancing sequence
  source 1..22: mol_type = other RNA
gcccgcgaggacccgcccgagc

SEQ ID NO: 29 - DNA, 201 bp, synthetic construct
  misc_feature 1..201: note = DCP1A promoter sequence
  source 1..201: mol_type = other DNA
gccatcgaggcaaaattcccccatctgcgtcagtcccctcaacttccgcctctacgcgggcctcgcggcagggcggtacgtcagccattctggtccgccgcgcgcacgctccgggcgccgggttccggcgttgtcagggtccgcggccctacgatgtgggcggtgtccaaggctgcgtagtggagcttgcaggctggagct

SEQ ID NO: 30 - DNA, 134 bp, synthetic construct
  misc_feature 1..134: note = Shortened DCP1A promoter sequence
  source 1..134: mol_type = other DNA
gccatcgaggcaaaattcccccatctgcgtcagtcccctcaacttccgcctctacgcgggcctcgcggcagggcggtacgtcagccattctggtccgccgcgcgcatgcgtagtggagcttgcaggctggagct

(19) United States
(12) Patent Application Publication
(10) Pub. No.: US 2023/0331818 A1
(43) Pub. Date: Oct. 19, 2023

(54) DESTABILIZED ARE 3'UTRs AS THERAPEUTICS FOR CANCER

(71) Applicants: Research Foundation of the City University of New York, New York, NY (US); President and Fellows of Harvard College, Cambridge, MA (US)

(72) Inventors: Olorunseun O. Ogunwobi, Yonkers, NY (US); Chidiebere U. Awah, Bronx, NY (US); Kevin Struhl, Weston, MA (US)

(21) Appl. No.: 18/176,871

(22) Filed: Mar. 1, 2023

Related U.S. Application Data
(60) Provisional application No. 63/315,368, filed on Mar. 1, 2022.

(51) Int. Cl. C07K 14/82; C12N 15/63

(52) U.S. Cl. CPC C07K 14/82; C12N 15/63

(57) ABSTRACT

A nucleotide that encodes for a destabilized RNA that corresponds to ERBB2 (erythroblastic oncogene B) or MYC oncogenes. The destabilization occurs in an adenylate-uridylate rich element (ARE) stabilizing motif. Without wishing to be bound to any particular theory, changing the stabilizing motifs of ERBB2 and MYC to destabilization motifs is believed to lead to the rapid degradation of their transcripts. Thus, in turn, renders the composition useful as a therapeutic for cancer.

### CROSS-REFERENCE TO RELATED APPLICATIONS

This application claims priority to, and is a non-provisional of, U.S. Patent Application 63/315,368 (filed Mar. 1, 2022), the entirety of which is incorporated herein by reference.

### STATEMENT OF FEDERALLY SPONSORED RESEARCH OR DEVELOPMENT

This invention was made with Government support under grant number U54CA221704 awarded by the National Cancer Institute. The government has certain rights in the invention.

### REFERENCE TO A SEQUENCE LISTING

This application contains a Sequence Listing in computer readable form. The computer readable form is incorporated herein by reference.

### BACKGROUND OF THE INVENTION

The subject matter disclosed herein relates to therapeutics for cancer treatment. Almost all therapies (chemo, immuno and endocrine therapies) used in treatment of cancers, including breast cancer show resistance. This represents a significant challenge to favorable clinical outcome in breast cancer as well other cancers. One of the key factors driving tumor resistance to therapies is oncogenic signals that drives rapid proliferation of cancer tissues. ERBB2 is implicated in many cancers and it is resistant to trastuzumab and other tyrosine kinase inhibitors.

The c-MYC is a basic helix loop helix (b-HLH) master transcription factor that bind the E-box sequences and is over expressed in >74% of human cancers. Breast cancers, lung cancers, brain tumors, prostate cancers, lymphomas, pediatrics cancers, pancreatic cancers, colorectal cancers, and ovarian cancers overexpress MYC. As at today, there is no direct clinically approved inhibitors of c-MYC. The lack of direct clinically approved inhibitors of c-MYC is a significant biological and clinical question that needs to be overcome for effective cancer treatment.

While attempts have been made to address these cancers, each treatment regiment comes with its own advantages and drawbacks. Additional treatment options are therefore desirable so as to provide medical practitioners with a wider range of treatment possibilities.

The discussion above is merely provided for general background information and is not intended to be used as an aid in determining the scope of the claimed subject matter.

### SUMMARY

This disclosure provides a nucleotide that encodes for a destabilized RNA that corresponds to ERBB2 (erythroblastic oncogene B) or MYC oncogenes. The destabilization occurs in an adenylate-uridylate rich element (ARE) stabilizing motif. Without wishing to be bound to any particular theory, changing the stabilizing motifs of ERBB2 and MYC to destabilization motifs is believed to lead to the rapid degradation of their transcripts. Thus, in turn, renders the composition useful as a therapeutic for cancer.

In a first embodiment, a composition of matter is provided. The composition of matter comprising a primary structure of SEQ ID NO: 19.

In a second embodiment, a composition of matter is provided. The composition of matter comprising sequential sequences, ordered from 5′ to 3′, of a first restriction sequence, a first polyA sequence, a promoter sequence, a nucleotide sequence with a primary structure of SEQ ID NO: 19, a second polyA sequence and a second restriction sequence.

In a third embodiment, a composition of matter is provided. The composition of matter comprising a primary structure of SEQ ID NO 18.

This brief description of the invention is intended only to provide a brief overview of subject matter disclosed herein according to one or more illustrative embodiments, and does not serve as a guide to interpreting the claims or to define or limit the scope of the invention, which is defined only by the appended claims. This brief description is provided to introduce an illustrative selection of concepts in a simplified form that are further described below in the detailed description. This brief description is not intended to identify key features or essential features of the claimed subject matter, nor is it intended to be used as an aid in determining the scope of the claimed subject matter. The claimed subject matter is not limited to implementations that solve any or all disadvantages noted in the background.

### BRIEF DESCRIPTION OF THE DRAWINGS

So that the manner in which the features of the invention can be understood, a detailed description of the invention may be had by reference to certain embodiments, some of which are illustrated in the accompanying drawings. It is to be noted, however, that the drawings illustrate only certain embodiments of this invention and are therefore not to be considered limiting of its scope, for the scope of the invention encompasses other equally effective embodiments. The drawings are not necessarily to scale, emphasis generally being placed upon illustrating the features of certain embodiments of the invention. In the drawings, like numerals are used to indicate like parts throughout the various views. Thus, for further understanding of the invention, reference can be made to the following detailed description, read in connection with the drawings in which:

FIG. 1 is a schematic diagram of one DNA sequence for treating cancer.

FIG. 2A is a RNA extract (erythroblastic oncogene B (ERBB2)) from MCF-7 cells. Stabilizing sequences are underscored.

FIG. 2B is a RNA extract from BT474 cells (ERBB2). Stabilizing sequences are underscored.

FIG. 2C is a RNA extract from T47D cells (ERBB2). Stabilizing sequences are underscored.

FIG. 3A is a RNA extract from MCF-7 cells (MYC). Stabilizing sequences are underscored.

FIG. 3B is a RNA extract from BR474 cells (MYC). Stabilizing sequences are underscored.

FIG. 3C is a RNA extract from T47D cells (MYC). Stabilizing sequences are underscored.

FIG. 3D is a RNA extract from MDA-MB231 cells (MYC). Stabilizing sequences are underscored.

FIG. 4A is a RNA extract from LNCAP cells (MYC). Stabilizing sequences are underscored.

FIG. 4B is a RNA extract from MDA Pca-2B cells (MYC). Stabilizing sequences are underscored.

FIG. 4C is a RNA extract from C4-2B cells (MYC). Stabilizing sequences are underscored.

FIG. 4D is a RNA extract from RWPE1-ex9 cells (MYC). Stabilizing sequences are underscored.

FIG. 5A is a RNA sequence showing a destabilized MYC RNA (changes relative to wildtype underscored).

FIG. 5B is a RNA sequence showing a destabilized ERBB2 RNA (changes relative to wildtype underscored).

FIG. 6A depicts a schematic of stable ERBB2 3′UTR.

FIG. 6B depicts a schematic of destabilized ERBB2 3′UTR.

FIG. 7A depicts a DNA sequence corresponding to the destabilized ERBB2 RNA. A first digestion enhancing sequence, a first restriction sequence, a first polyA sequence and a promoter sequence are sequentially disposed 5′ of the DNA sequence. A second polyA sequence, a second restriction sequence and a second digestion enhancing sequence are sequentially disposed 3′ of the DNA sequence.

7B depicts a DNA sequence corresponding to the destabilized MYC RNA. A first digestion enhancing sequence, a first restriction sequence, a first polyA sequence and a promoter sequence are sequentially disposed 5′ of the DNA sequence. A second polyA sequence, a second restriction sequence and a second digestion enhancing sequence are sequentially disposed 3′ of the DNA sequence.

FIG. 7C depicts a DNA sequence corresponding to the destabilized ERBB2 RNA.

FIG. 7D depicts a DNA sequence corresponding to the destabilized MYC RNA.

FIG. 7E depicts a DCP1A promoter sequence and a shortened DCP1A promotor sequence.

FIG. 8 is a schematic diagram illustrating one method of incorporating a DNA sequence into a plasmid vector.

FIG. 9A shows BT474 breast cancer cells (trastuzumab resistant HER2+ breast cancer cells) experienced severe morphology disruption after four days. Treatment was with the plasmid vector containing destabilized 3′ UTR ERBB2. Two wild type controls (BT474 WT and BT474 WT vector (empty)) are shown for comparison.

FIG. 9B shows BT474 breast cancer cells experienced significant cell death after eight days. Treatment was with the plasmid vector containing destabilized 3′ UTR ERBB2. Two wild type controls (BT474 WT and BT474 WT vector (empty)) are shown for comparison.

FIG. 10A is a graph showing cell viability of BT474 cells after treatment with destabilized 3′ UTR ERBB2.

FIG. 10B is a Western blot showing ERBB2 and GAPDH expression after treatment of BT474 cells with destabilized 3′ UTR ERBB2.

FIG. 10C is a bar chart that quantifies ERBB2 expression normalized against GAPDH after treatment of BT474 cells with destabilized 3′ UTR ERBB2.

FIG. 11A is a graph showing cell viability of MDA MB231 cells after treatment with destabilized 3′ UTR MYC.

FIG. 11B is a Western blot showing MYC and GAPDH expression after treatment of MDA MB231 cells with destabilized 3′ UTR MYC.

FIG. 11C is a bar chart that quantifies MYC expression normalized against GAPDH after treatment of MDA MB231 cells with destabilized 3′ UTR MYC.

FIG. 12A is a graph showing cell viability of C4 B2 cells after treatment with destabilized 3′ UTR MYC.

FIG. 12B is a Western blot showing MYC and GAPDH expression after treatment of C4 B2 cells with destabilized 3′ UTR MYC.

FIG. 12C is a bar chart that quantifies MYC expression normalized against GAPDH after treatment of C4-2B cells with destabilized 3′ UTR MYC.

FIG. 13A is a graph showing cell viability of NCI H1975 cells after treatment with destabilized 3′ UTR ERBB2.

FIG. 13B is a Western blot showing ERBB2 and GAPDH expression after treatment of NCI H1975 cells with destabilized 3′ UTR ERBB2.

FIG. 13C is a bar chart that quantifies ERBB2 expression normalized against GAPDH after treatment of NCI H1975 cells with destabilized 3′ UTR ERBB2.

FIG. 14A is a bar graph showing tumor volume (BT474 cells) in mice after treatment with various desARE ERBB2 clones.

FIG. 14B is a graph of the probability of survival of the mice of FIG. 14A.

FIG. 15A is a bar graph showing tumor volume (NCI H1975 cells) in mice after treatment with various desARE ERBB2 clones.

FIG. 15B is a bar graph showing number of liver metastasis sites found for the mice of FIG. 15A.

FIG. 16A is a bar graph showing number of tumor metastatic sites found in mice after implantation with MDA-MB231 cells.

FIG. 16B is a graph of the probability of survival of the mice of FIG. 16A.

### DETAILED DESCRIPTION OF THE INVENTION

This disclosure provides a therapeutic agent for ERBB2 (erythroblastic oncogene B) in cancers including breast cancer, and in trastuzumab resistant breast cancers, triple negative breast cancer, colon cancer, lung cancer and osteosarcoma where ERBB2 oncogene is a key driver of cancer pathogenesis. This disclosure also provides a therapeutic agent for MYC oncogene overexpression for which there is no therapy in over sixteen major human tumors. MYC oncogene is overexpressed in breast adenocarcinoma, lung squamous carcinoma, lung adenocarcinoma, hepatocellular carcinoma, esophagus carcinoma, hepatocellular carcinoma, gastric carcinoma, colon adenocarcinoma, pancreatic adenocarcinoma, ovarian adenocarcinoma, bladder adenocarcinoma, uterine carcinosarcoma, prostate adenocarcinoma, endometrial adenocarcinoma, acute myeloid leukaemia, Diffuse B cell lymphoma and Osteosarcoma.

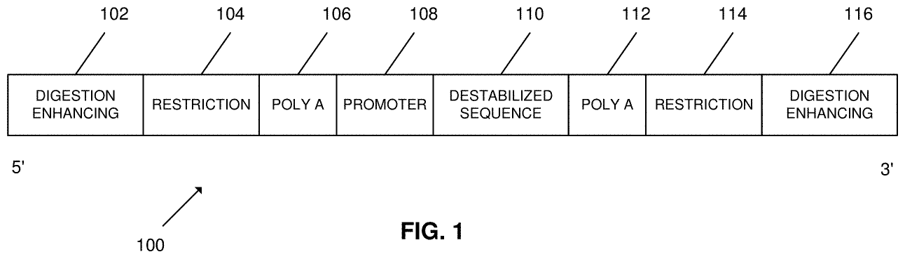

Referring to FIG. 1, a sequence 100 is show which comprises a first digestion enhancing sequence 102, a first restriction sequence 104, a first polyA sequence 106, a promoter sequence 108, a destabilized sequence 110 (the disclosed destabilized ARE 3′UTRs), a second polyA sequence 112, a second restriction sequence 114 and a second digestion enhancing sequence 116. In one embodiment, the sequence 100 is a DNA sequence that is incorporated into a plasmid vector.

The first digestion enhancing sequence 102 is located at the 5′ end and may be selected from known sequences. In one embodiment, the first digestion enhancing sequence 102 is between five and one hundred residues. In another embodiment, the first digestion enhancing sequence 102 is between five and fifty residues. In one embodiment, both the first digestion enhancing sequence 102 and the digestion enhancing sequence 116 are the same. In one embodiment, the first digestion enhancing sequence 102 is GGACCCGCCCGAGC (SEQ ID NO: 26). In another embodiment, the first digestion enhancing sequence 102 is GGGCCGGCCCCGCCG (SEQ ID NO: 27). In yet another embodiment, the first digestion enhancing sequence 102 is

(SEQ ID NO: 28)
GCCCGCGAGGACCCGCCCGAGC.

The first restriction sequence 104 may be selected from known sequences. Suitable examples include a BstB1 restriction site, a BamH1 restriction site, a Xba1 restriction site, a Apa1 restriction site, a PspOM1 restriction site, and the like. In one embodiment, the first restriction sequence 104 has between four and seven residues. In one embodiment, both the first restriction sequence 104 and the second restriction sequence 114 are different.

The first polyA sequence 106 generally contains between five and eleven residues. The first polyA sequence 106 and the second polyA sequence 112 may be the same or different. The first polyA sequence functions to stop the RFP transcription.

The promoter sequence 108 may be selected from known sequences. Suitable examples include a DCP1A promoter, a DCP2 promoter and a ZFP36 promoter. In one embodiment, the promoter sequence 108 is between one hundred forty and one hundred seventy residues. In another embodiment, promoter sequence 108 is between one hundred fifty and one hundred sixty residues.

The destabilized sequence 110 is based on RNAs that are associated with cancer but wherein the resulting RNA is less stable than the wildtype RNA found in cancer cells. For example, the 3′UTR (untranslated regions) of the oncogenes ERBB2, MYC in breast and prostate cancer cell lines is enriched with poly UUUUUU sequences which are mRNA adenylate-uridylate rich element (ARE) stabilizing motif. Without wishing to be bound to any particular theory, changes in the stabilizing motifs of ERBB2 and MYC to destabilization motifs are believed to lead to the rapid degradation of their transcripts. Thus, in turn, renders sequence 100 useful as a therapeutic for cancer. The details of the destabilized sequence 110 are disclosed elsewhere in this specification.

The second polyA sequence 112 contains between five and eleven residues. The first polyA sequence 106 and the second polyA sequence 112 may be the same or different. The second polyA sequence 112 functions to stop transcription.

The second restriction sequence 114 may be selected from known sequences. Suitable examples include a BstB1 restriction site and a BamH1 restriction site. In one embodiment, both the first restriction sequence 104 and the second restriction sequence 114 are different.

The second digestion enhancing sequence 116 may be selected from known sequences. In one embodiment, the first digestion enhancing sequence is between 5 and 50 residues. In one embodiment, the second digestion enhancing sequence 116 is GGACCCGCCCGAGC (SEQ ID NO: 26). In another embodiment, the second digestion enhancing sequence 116 is GGGCCGGCCCCGCCG (SEQ ID NO: 27). In yet another embodiment, the second digestion enhancing sequence 116 is

(SEQ ID NO: 28)
GCCCGCGAGGACCCGCCCGAGC.

By way of illustration, total RNA was extracted from breast cancer cell lines: BT474, MDA MB231, T47D and MCF7. Total RNA was also extracted from the prostate cancer cell lines: LNCAP, MDA PCa2B, C4-2B and RWPE1 ex9. Samples of cDNA were made from these total RNA using the Qiagen reverse transcription kit (Catalog no: 205311). RT-PCR was performed using the cDNA according to the Qiagen manufacturers protocol using the following primer sequences:

ERBB2 forward:
(SEQ ID NO: 20)
GCCTCGTTGGAAGAGGAACA
ERBB2 reverse:
(SEQ ID NO: 21)
AGAGCCACCCCCAGACATAG
MYC reverse:
(SEQ ID NO: 23)
GGATTGAAATTCTGTGTAACTGC
DCP1A Forward
(SEQ ID NO: 24)
CCCTCAACTTCCGCCTCTAC promoter
DCP1A Reverse
(SEQ ID NO: 25)
CAGCCTGCAAGCTCCACTAC promoter
MYC Forward
(SEQ ID NO: 22)
CCTCACAACCTTGGCTGAGT

The PCR cycles were as follows: (Expected ERBB2 3′UTR amplicon size=464 bp, Expected MYC 3′UTR amplicon size=246 bp). PCR Cycle for cDNA synthesis: , 94 for 2 mins, 94° C. for 15 sec, 55° C. for 30 sec, 68-72° C. for 1 min, Step 2-4 for 40 cycles, 4° C. hold. PCR set up: 38.1 μL of H₂O, 1.5 μL , of 50 mM Mgcl, 1 μL of 10 mM DNTP, 1 μL ERBB2/MYC Forward primers, 1 μL ERBB2/MYC Reverse primers, 2 of cDNA (200 ng), 1 μL of Taq polymerase. Amplicon was separated on 2% agarose gel. The band sizes of ERBB2 464 bp and 264 bp for MYC were excised under UV light and then extracted with Qiagen gel extraction kit (Catalog no: 28706X4) according to manufacturer's protocol. The amplicon was sequenced by Sanger Sequencing method.

To find the stabilizing ARE motif on the 3′UTR of ERBB2 and MYC genes, the cDNA sequences were translated to mRNA sequences using conventional web software.

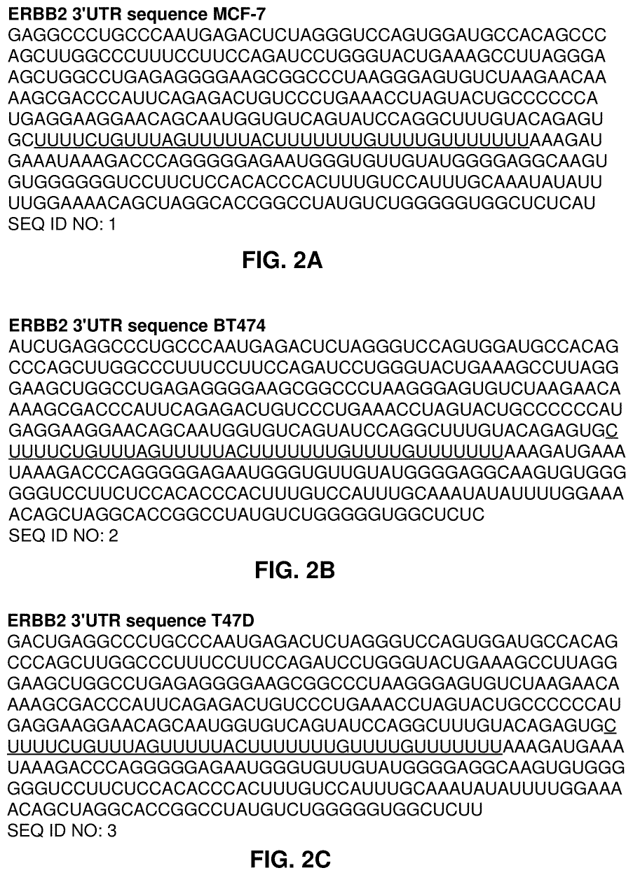

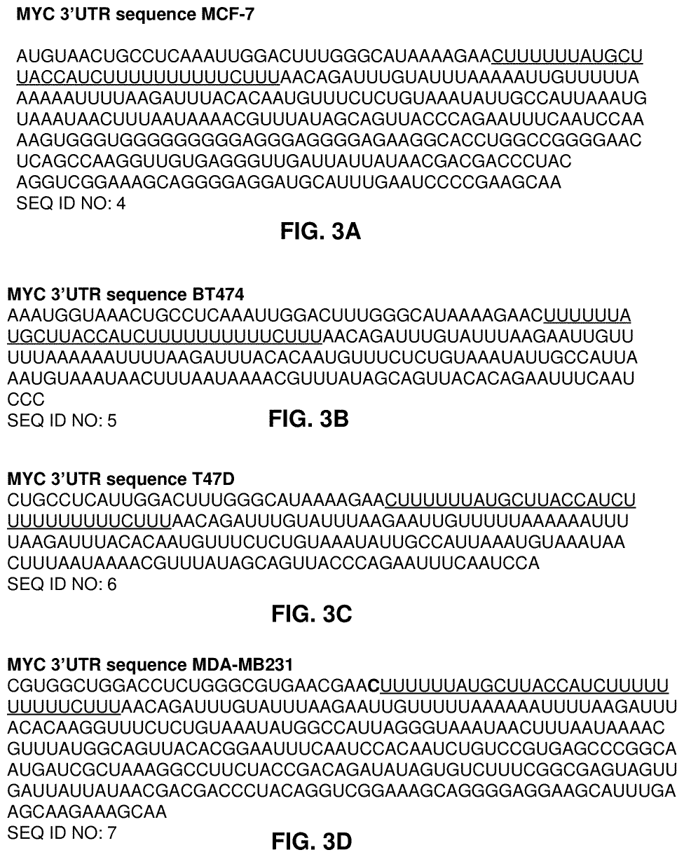

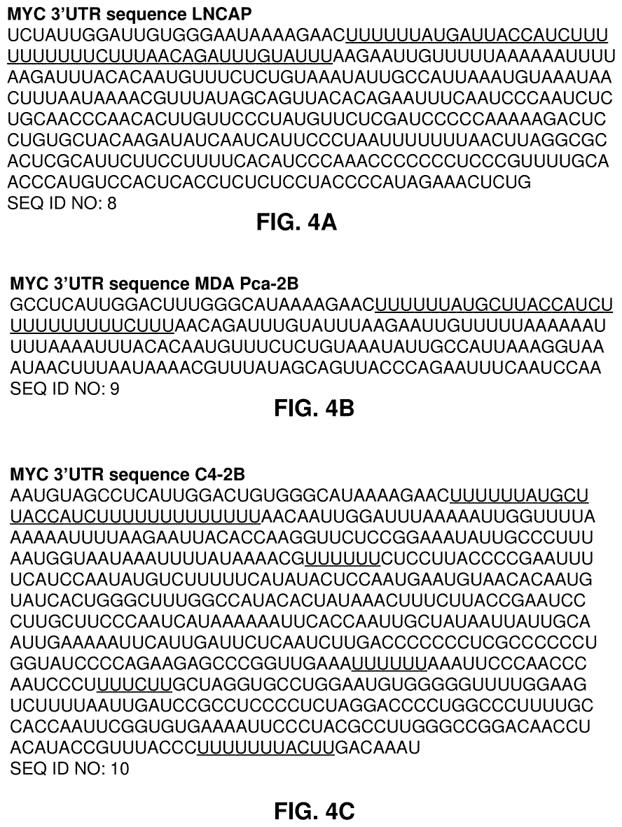

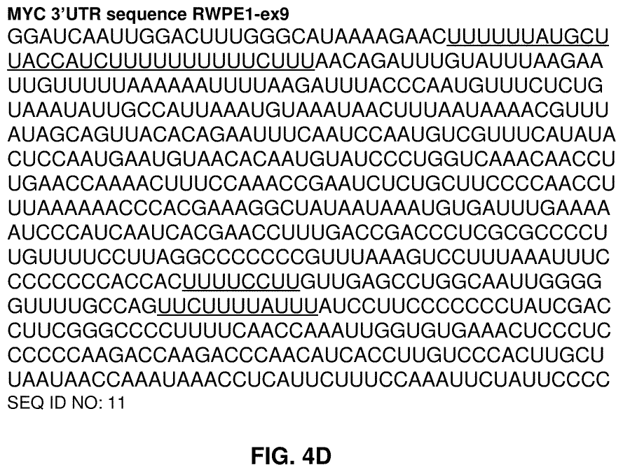

TABLE 1

| Cell line | Cancer | Oncogene | FIG. |
|---|---|---|---|
| MCF7 | Breast | ERBB2 | FIG. 2A (SEQ ID NO: 1) |
| BT474 | Breast | ERBB2 | FIG. 2B (SEQ ID NO: 2) |
| T47D | Breast | ERBB2 | FIG. 2C (SEQ ID NO: 3) |
| MCF7 | Breast | MYC | FIG. 3A (SEQ ID NO: 4) |
| BT474 | Breast | MYC | FIG. 3B (SEQ ID NO: 5) |
| T47D | Breast | MYC | FIG. 3C (SEQ ID NO: 6) |
| MDA MB231 | Breast | MYC | FIG. 3D (SEQ ID NO: 7) |
| LNCAP | Prostate | MYC | FIG. 4A (SEQ ID NO: 8) |
| MDA PCa2B | Prostate | MYC | FIG. 4B (SEQ ID NO: 9) |
| C4-2B | Prostate | MYC | FIG. 4C (SEQ ID NO: 10) |
| RWPE1 ex9 | Prostate | MYC | FIG. 4D (SEQ ID NO: 11) |

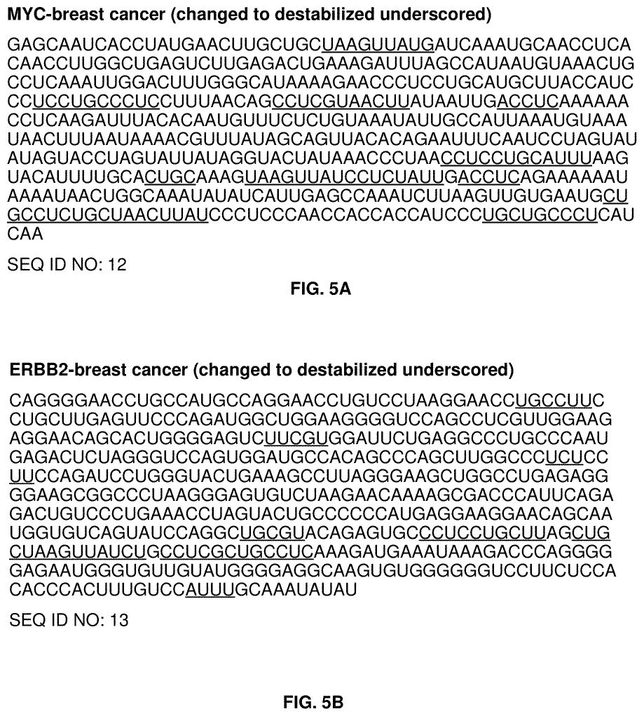

To design the destabilized 3′UTR of ERBB2 and MYC, the consensus stabilized motifs (e.g. UUUUU, UUGCAUGG, CCUUACAC) in the mRNA were selectively replaced with destabilized ARE consensus motifs. The resulting RNA sequences are shown in FIG. 5A (MYC) and FIG. 5B (ERBB2), corresponding to SEQ ID NO: 12 and SEQ ID NO: 13 respectively.

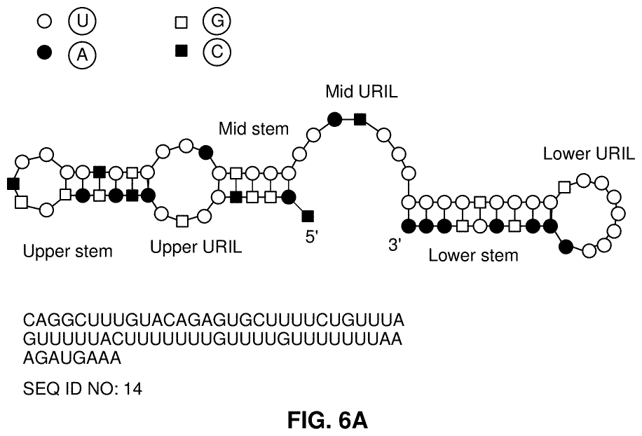

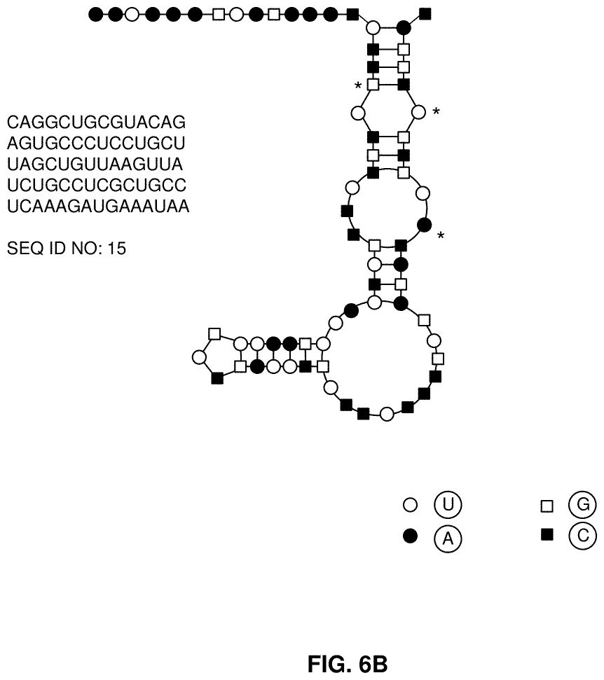

Some residues on the 3′ end of the 3′UTR were modified to increase stability. Torabi (Science, 371:6529, Jan. 7, 2021), used structural biology and biochemical assays to delineate residues of helices of the nucleic acids on the polyA tail of 3′UTR mRNA that determines their strong, moderate stability as well as rapid degradation. Based on this, the loop structures of the stabilized (FIG. 6A, SEQ ID NO: 14) and destabilized (FIG. 6B, SEQ ID NO: 15) 3′UTR of ERBB2 and MYC (BMC Bioinformatics, 11:129, 2010) were analyzed. The stabilized ERBB2 ARE structures have stability determining U-A on the lower loop stem as well as on the lower URIL. Using the mutated residues from Torabi, that confers moderate degree stability as an example. In designing the destabilizing ERBB2 and MYC 3′UTR (the M5 (C-U), M6 (U-C) and M11 (A-U) were changed with all residues marked with asterisks in FIG. 6B). Changes in these residues have a remarkable impact on the stability of transcript up to 120 mins.

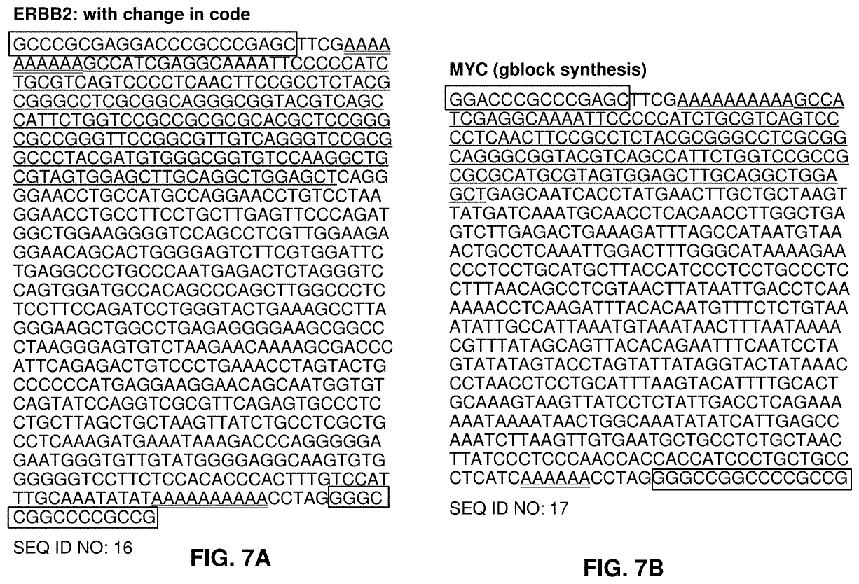

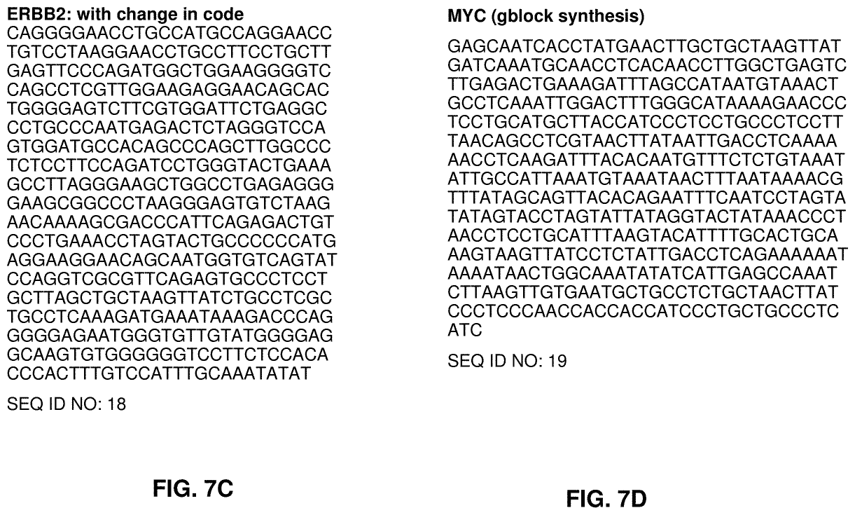

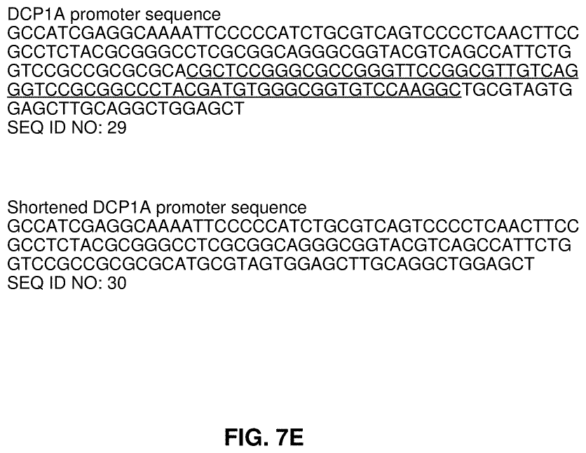

Having established the structure of stabilized ERBB2 and MYC 3′UTR and mutated residue of destabilized ERBB2 and MYC 3′UTR that increased their stability, these destabilized constructs were incorporated with an upstream 5′ BstB1 restriction site followed by a polyA sequence to stop the RFP transcription and then followed by a DCP1A promoter. At the 3′ end of the RNA sequences, a polyA sequence was added followed by BamH1 restriction site as shown in FIG. 7A (ERBB2) and FIG. 7B (MYC), corresponding to SEQ ID NO: 16 and SEQ ID NO: 17, respectively.

In FIG. 7A, the 5′ sequence contains a first digestion enhancing sequence (boxed), a Bstb1 sequence (TTCGAA) followed by a polyA sequence (AAAAAAAA, double underscored), a DCP1A promoter sequence (single underscore in FIG. 7A, SEQ ID NO: 29 in FIG. 7E) the destabilized 3′UTR sequence (no underscoring or boxing), followed by a polyA sequence (double underscored), a BamH1 restriction sequence (CCTAG) and a second digestion site (boxed). The 5′ Bstb1 site as well as the 3′ BamH1 sites enables the cloning into the sp6 vector. The upstream polyA sequence is to stop the transcription of the RFP from the Sp6 vector. The polyA tail at the 3′end is to stop the transcription of DCP1A promoter. FIG. 7C depicts the same DNA sequence with the first digestion enhancing sequence, the first restriction sequence, the polyA sequence, the promoter sequence, the second restriction sequence, and the second digestion sequence removed, corresponding to SEQ ID NO: 18.

In FIG. 7B, the 5′ sequences contain first digestion enhancing sequence (boxed), a BstB1 restriction site (TTCGAA), a polyA sequence (double underscore), a shortened DCP1A promoter sequence (single underscore in FIG. 7B, SEQ ID NO: 30 in FIG. 7E), the destabilized 3′UTR sequence (no underscore), followed by a polyA sequence and then a BamH1 restriction sequence (double underscored). FIG. 7D depicts the same DNA sequence with the first enhancing digestion sequence, the first restriction sequence, the polyA sequence, the promoter sequence, the second restriction sequence, and the second digestion sequence, removed, corresponding to SEQ ID NO: 19.

Referring to FIG. 7E, the DCP1A promoter sequence (SEQ ID NO: 29) was shortened by removing the underscored residues, thereby resulting in the shortened DCP1A promoter sequence (SEQ ID NO: 30).

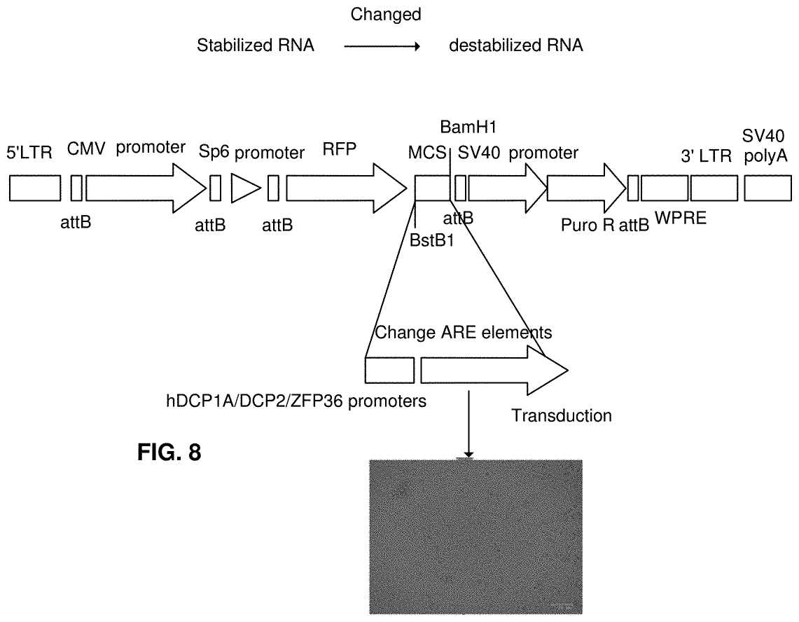

The fully destabilized 3′UTR of ERBB2 and MYC were synthesized as gblock from IDT Cloning of the destabilized 3′UTR of ERBB2 and MYC into Sp6 vector. See FIG. 8.

The destabilized 3′UTR of ERBB2 and MYC were cloned into pLenti-CMVSP6-nEGFP-SV40-PURO (Addgene: #138364) as described below.

Miniprep of vector: A stab of the vector was inoculated into LB media and grown over night shaking at 250 rpm at 37° C. The plasmid vector gDNA was extracted using the Qiagen midikit (Cat no: 12943).

Digest of vector with BstBi and BamH1: The vector was digested in BstBi and BamH1 at 37° C. overnight using NEB buffer 2.1 1 μl (400 ng) of vector gDNA, 1 μl of Bstb1 and Bamh1 respectively 3 μl of buffer, 25 μl of H₂O.

Gel extraction of vector: The digested vector was gel extracted using Qiagen gel extraction kit (Cat no28704).

Digest insert with BstB1 and BamH1: The synthetic gblock containing the destabilized ERBB2 and MYC 3′UTR were digested with BstB1 and BamH1 in NEB buffer 2.1 for 1 hr at 37° C., 1 μl (200 ng) of gblock DNA, 1 ul of Bstb1 and Bamh1 respectively 3 μl of buffer, 25 μl of H₂O.

Gel extraction of insert: The digested synthetic gblock containing the destabilized ERBB2 and MYC 3′UTR was gel extracted using Qiagen gel extraction kit (Cat no: 28704).

Ligation: T4 ligase buffer 2 μl, Plasmid vector 0.5 μl, Insert (digested synthetic gblock) 4 μl Nuclease free water 20 μl, T4 ligase 1 μl (NEB), At 22° C. for 3 hrs.

Transformation: competent E-coli (NEB 5 alpha Cat no: C298H) were transformed using SOC media, 25 ul of each cells was incubated on ice with 5 ul of the ligation mix for 30 mins. After 30 mins was heat shocked in water bath at 42° C. for 30 sec. Tubes were placed back on ice for 2 mins and 900 ul of SOC media was added and then incubated at 37° C. shaking for 1 hr at 250 rpm. After which they were plated on LB Agar plate containing ampicillin and the plate was incubated overnight at 37° C.

Colony picking and Miniprep: Colonies were picked with pipette tips and inoculated into 5 ml LB media containing Ampicillin and grown overnight at 37° C. shaking at 250 rpm. The pellets were spun down and gDNA was extracted using the Qiagen midikit (Cat no: 12943) and using a nanodrop machine the gDNA were quantified.

Colony PCR with ERBB2 3′UTR primers and DCP1A promoter primers: PCR was performed with ERBB2 primers and DCP1A primers using the PCR cycle described elsewhere in this specification.

Gel extraction: The colony PCR products of ERBB2 3′UTR and DCP1a promoters was gel extracted using Qiagen gel extraction kit (Cat no: 28704).

Sanger sequencing: The cloned synthetic gblock amplicons of ERBB2 3′UTR were sequenced using the primers described elsewhere in this specification by Sanger Sequencing.

Gibson Assembly: While the destabilized ERBB2 3′UTR was successfully incorporated into the vector by ligation (sticky end cloning), a GIBSON Assembly was used with MYC 3′UTR. The GIBSON Assembly was successful in cloning MYC and ERBB2 destabilized 3′UTR synthetic constructs into the plasmid vector. See U.S. Provisional patent application 63/315,368 for additional details. Each of the ERBB2 clones includes random variations that naturally occurs during cloning which impacts their performance. Likewise, similar natural variations are present in each of the MYC clones.

TABLE 2

| Clone | Vector incorporation method |
|---|---|
| desARE 3′UTR ERBB2-1 | Ligation |
| desARE 3′UTR ERBB2-2 | Ligation |
| desARE 3′UTR ERBB2-3 | Ligation |
| desARE 3′UTR ERBB2-4 | Ligation |
| desARE 3′UTR ERBB2-30 | Gibson Assembly |
| desARE 3′UTR MYC1-14 | Gibson Assembly |
| desARE 3′UTR MYC1-18 | Gibson Assembly |
| desARE 3′UTR MYC2-3 | Gibson Assembly |

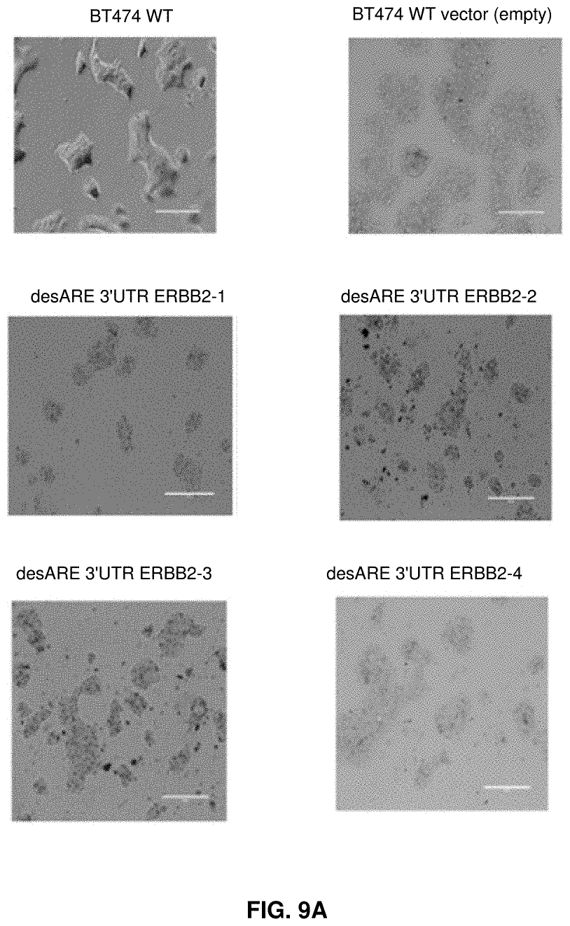

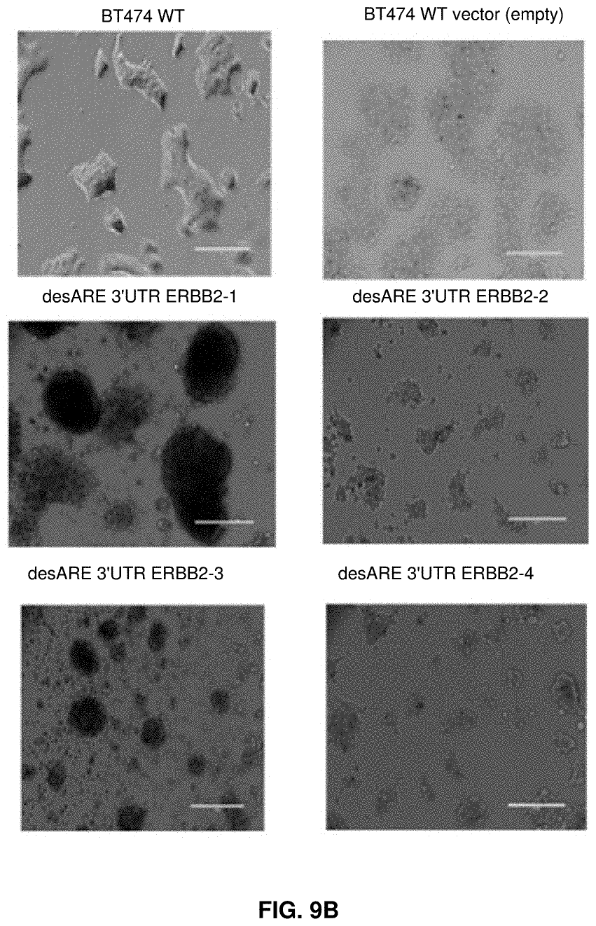

To introduce the destabilized 3′UTR into the cancer cells, constructs were electroporated into 200,000 to 500,000 cells with 50 ng of plasmid containing the constructs using the BioRad electroporation system. The preset mammalian protocol set was used for 293T cells and pulsed the cells in a cuvette 2×. Cells were then seeded into six well plates and viewed for morphology and red fluorescent protein (RFP) expression in 24 hrs. After 24 hrs the cells expressed RFP. Cell morphology of BT474 cells is depicted in FIG. 9A and FIG. 9B.

FIG. 9A depicts two controls (BT474 WT and BT474 vector (empty)). Four different images are shown after treatment with destabilized ERBB2 showing degradation of the tumor after four days. In FIG. 9B four additional images are shown showing the desARE3′UTR ERBB2 killed up to 90% of the cancer cells after eight days.

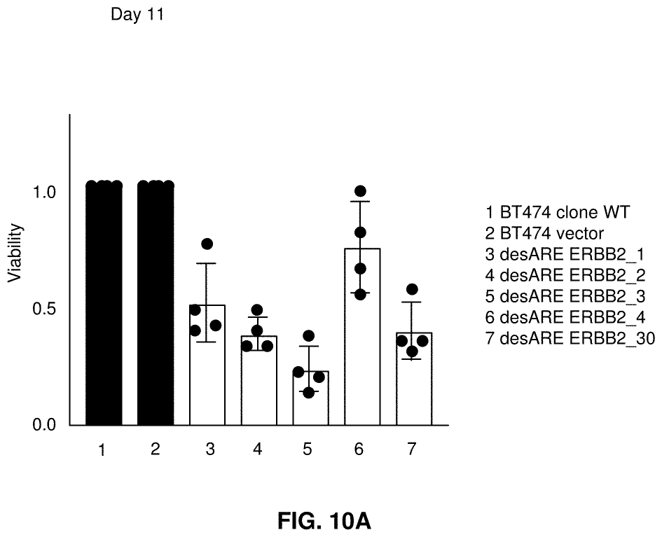

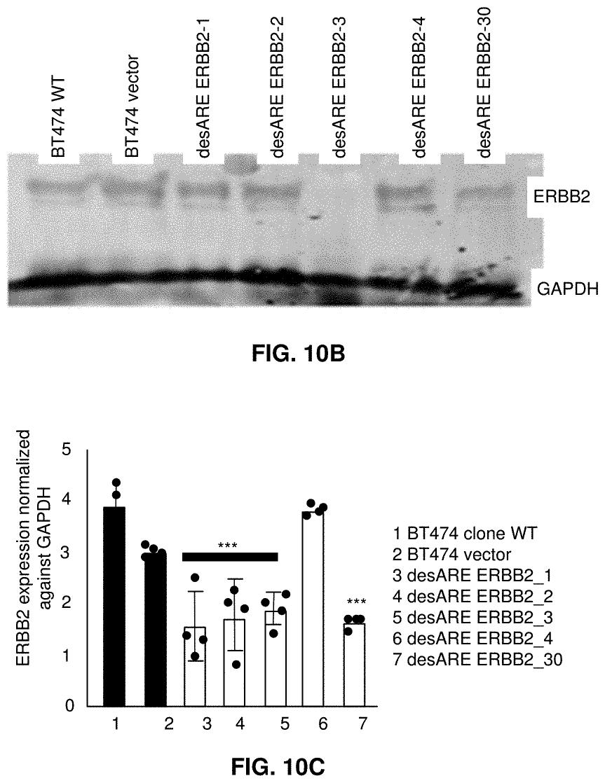

FIG. 10A depicts cell viability data of BT474 cell lines after eleven days of treating with 10 μg per μL of destabilized 3′ UTR ERBB2 plasmid vector (n=2, paired two tailed T-test (**P<0.01, ****P<0.0001, ns=0.084504)). FIG. 10B is a Western blot showing ERBB2 and GAPDH expression after treatment with destabilized 3′ UTR ERBB2 (n=3). FIG. 10C is a bar chart that quantifies the ERBB2 expression using qPCR normalized against GAPDH (Paired two tailed T-test (**P<0.01, ****P<0.0001, ns=0.084504, n=2)).

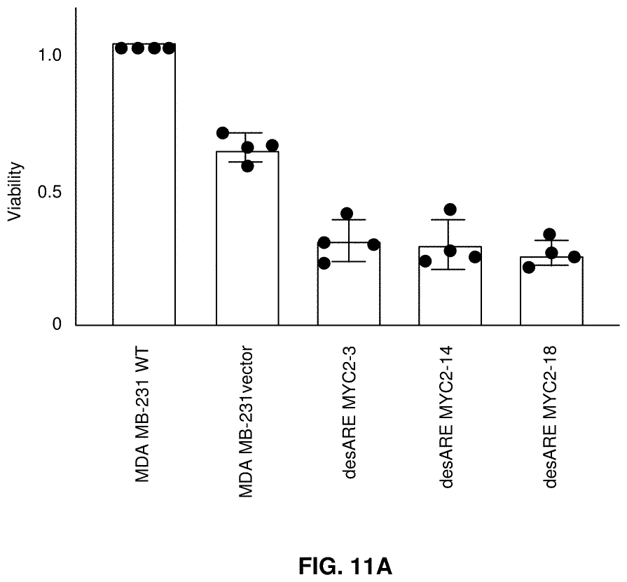

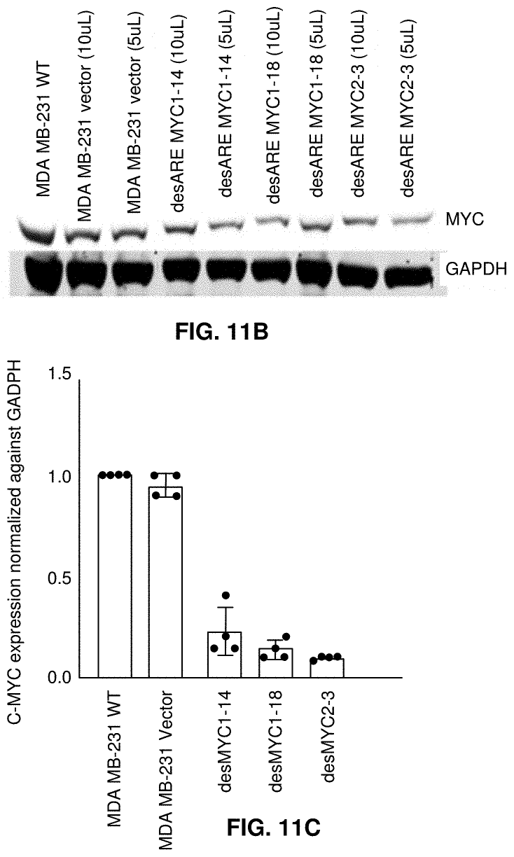

FIG. 11A depicts cell viability data of MDA MB231 cell lines after treating with 450 ng per μL of destabilized 3′ UTR MYC plasmid vector (n=6, **P=0.001(WT vs vector), ***P<0.0001 (WT vs 3′UTRMYC2-3, 1-18, 1-14)). FIG. 11B is a Western blot showing MYC and GAPDH expression after treatment with destabilized 3′ UTR MYC (n=4). FIG. 11C is a bar chart that quantifies the MYC expression using qPCR normalized against GAPDH (****P<0.00001(WT vs 3′UTR MYC1-18), ***P<0.0001(WT vs BT474 3′UTRMYC1-14, n=5).

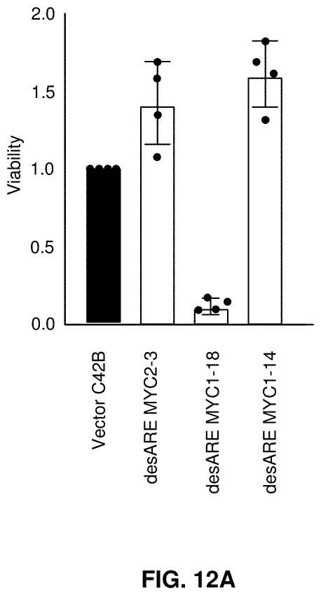

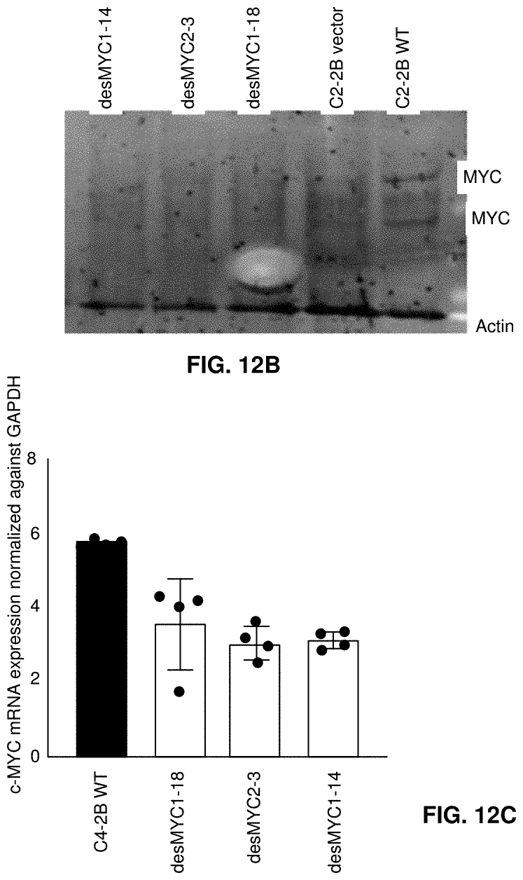

FIG. 12A depicts cell viability data of the C4-B cell line after treating with 10 μg per μL of destabilized 3′ UTR MYC plasmid vector. FIG. 12B is a Western blot showing MYC and GAPDH expression after treatment with destabilized 3′ UTR MYC of C4 2B cell lines. FIG. 12C is a bar chart that quantifies the MYC expression using qPCR normalized against GAPDH.

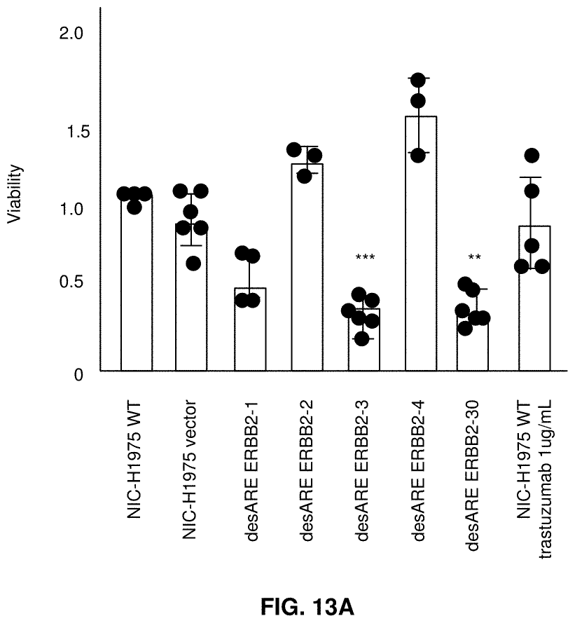

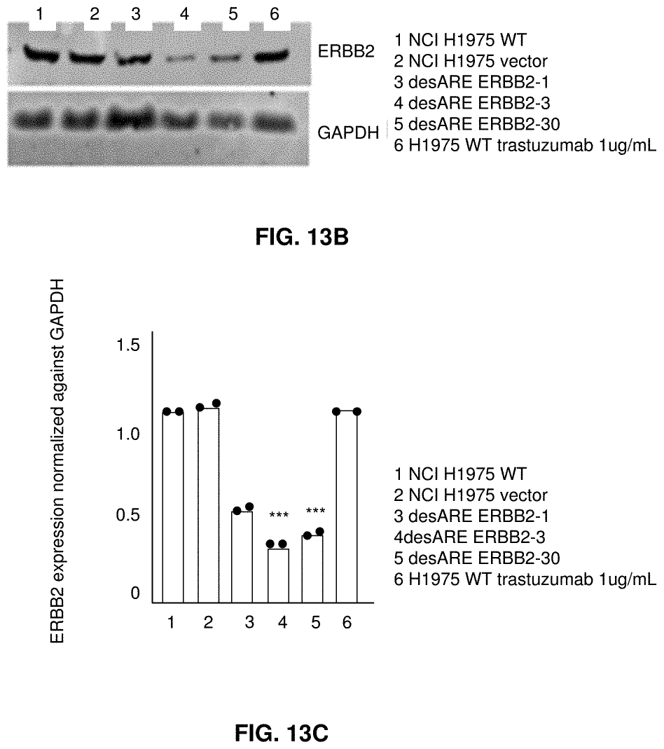

FIG. 13A depicts cell viability data of NCI H1975 cell lines (EGFRT790M HER2+ Osimertinib resistant non-small cell lung cancer) after treating with 10 μg per μL of destabilized 3′ UTR ERBB2 plasmid vector (n=2, Paired two tailed T-test (***P value=0.0013 desARE3′UTR ERBB2-3, **P value=0.03 desARE3′UTR ERBB2-30)). FIG. 13B is a Western blot showing ERBB2 and GAPDH expression after treatment with destabilized 3′ UTR ERBB2 (n=2). FIG. 13C is a bar chart that quantifies the ERBB2 expression using qPCR normalized against GAPDH (***P value=0.007569 desARE3′UTR ERBB2-3, ***P value=0.001123 desARE3′UTR ERBB2-30, n=2).

### Animal Studies

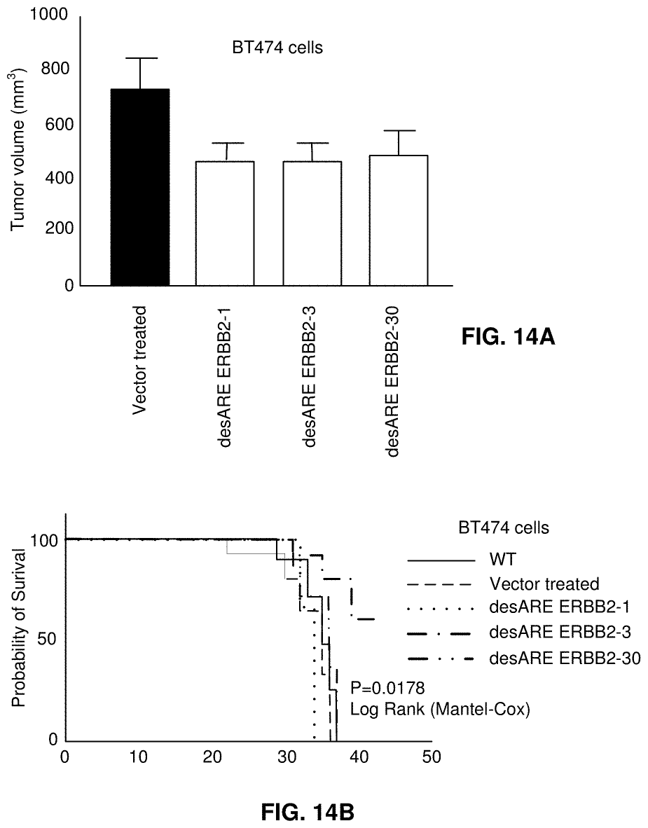

Referring to FIG. 14A and FIG. 14B, animal studies were conducted on the ERBB2 clones using twenty-five NSG mice. Mammary fat pads were implanted with five million BT474 cells (ERBB2+ trastuzumab resistant breast cancer). On day fourteen, huge tumors were observed. On day fifteen, the mice were randomized. From day sixteen 20 μg of desARE ERBB2-1, desARE ERBB2-2 or desARE ERBB2-30 was administered 2× every week (6 pm first dose, 6 am the second dose) till 57 days. The tumor volumes were measured at the conclusion of the experiment at fifty-seven days (FIG. 14A). In FIG. 14B, the survival data of the mice is shown. Mice treated with desARE ERBB2-3 had exceptional survivors.

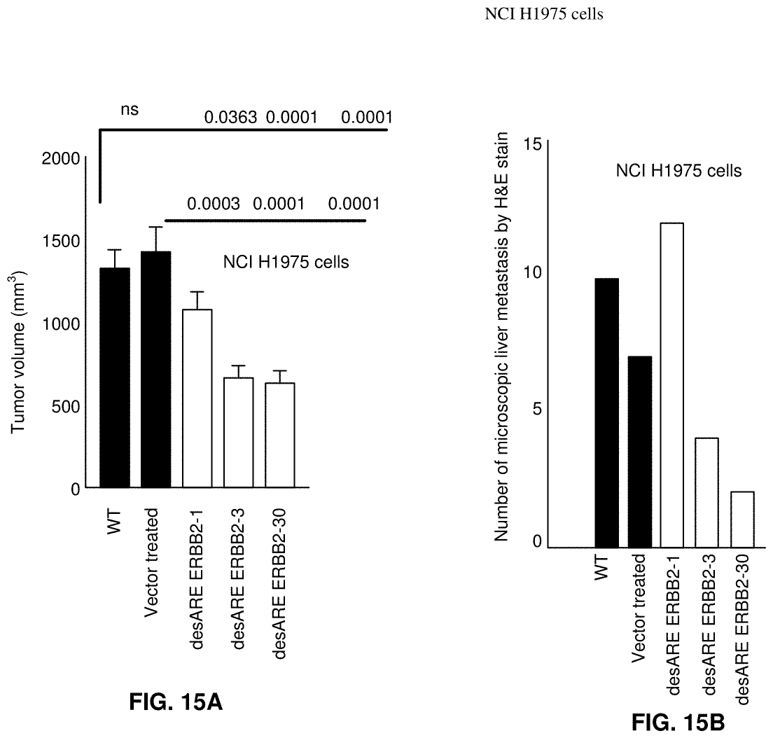

Referring to FIG. 15A and FIG. 15B, five million NCI H1975 cells (ERBB2+ osimertinib resistant EGFRT790M non-small cell lung cancer) were flank xenograph implanted into twenty five mice. On day thirty-five, huge tumors were observed. On day thirty-six the mice were randomized. From days thirty-seven to fifty-seven 2× per every 12 hrly (8 pm first dose, 8 am second dose), the mice received daily injections of 20 μg of desARE ERBB2-1, desARE ERBB2-2 or desARE ERBB2-30. Dosing break was observed on day fifty. The tumor volumes were measured at the conclusion of the experiment at seventy-seven days (FIG. 15A). In FIG. 15B, the number of liver metastatsis tumors is displayed in a bar chart as determined by H&E staining.

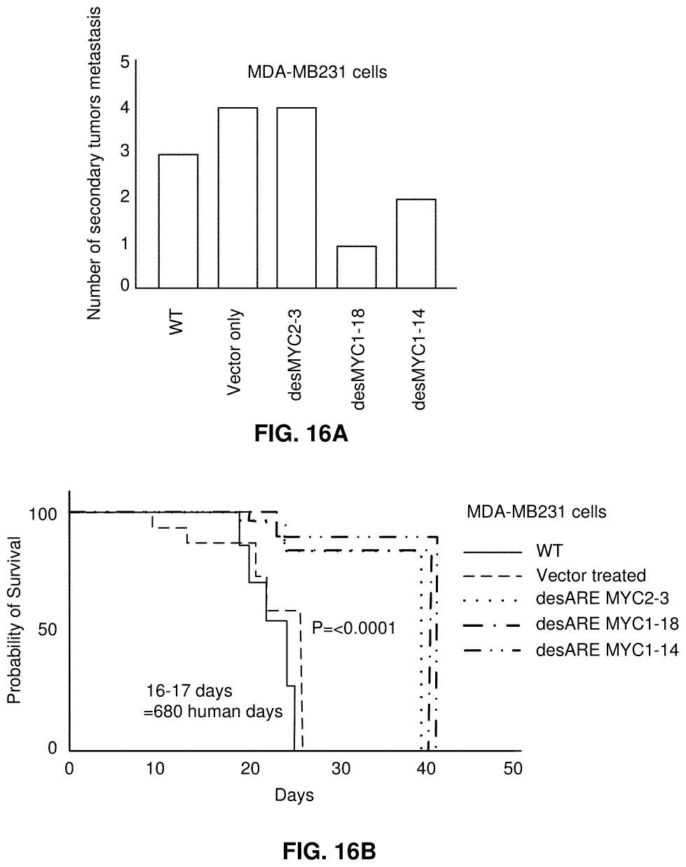

Referring to FIG. 16A and FIG. 16B, mammary fat pads of thirty mice were implanted with ten million MDA-MB231 cells. By day twenty-seven huge tumors were observed. On day twenty-eight, the mice were randomized. From day thirty to thirty-one, the mice received daily injections of 20 μg of desARE MYC2-3, desARE MYC2-18, desARE MYC2-14. On day thirty-two, a dosing break was permitted. From day thirty to day sixty-eight, a dosing break was observed on day 32 The experiment concluded at day sixty-eight. FIG. 16A depicts the number of secondary tumor metastatic sites while FIG. 16B depicts the probability of survival of the mice.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A composition of matter comprising a primary structure of SEQ ID NO: 19.

2. A plasmid vector comprising the composition of matter as recited in claim 1.

3. The composition of matter as recited in claim 1, wherein the composition of matter consists of the primary structure of SEQ ID NO: 19.

4. A composition of matter comprising sequential sequences, ordered from 5′ to 3′, of a first restriction sequence, a first polyA sequence, a promoter sequence, a nucleotide sequence with a primary structure of SEQ ID NO: 19, a second polyA sequence and a second restriction sequence.

5. The composition of matter as recited in claim 4, wherein the composition of matter consists of the first restriction sequence, the first polyA sequence, the promoter sequence, the nucleotide sequence with a primary structure of SEQ ID NO: 19, the second polyA sequence and the second restriction sequence.

6. A plasmid vector comprising the composition of matter as recited in claim 5.

7. The composition of matter as recited in claim 4, wherein the promotor sequence is a DCP1A sequence selected from a group consisting of SEQ ID NO: 29 and SEQ ID NO: 30.

8. The composition of matter as recited in claim 4, wherein the promotor sequence is selected from a group consisting of a DCP1A promoter, a DCP2 promoter and a ZFP36 promoter.

9. The composition of matter as recited in claim 4, wherein the first restriction sequence and the second restriction sequence are different.

10. The composition of matter as recited in claim 4, wherein the first restriction sequence is a BstB1 sequence.

11. The composition of matter as recited in claim 4, wherein the second restriction sequence is a BamH1 sequence.

12. The composition of matter as recited in claim 4, wherein the first polyA sequence and the second polyA sequence each contains between five and eleven residues.

13. The composition of matter as recited in claim 5, wherein the first restriction sequence has between four and seven residues, the first polyA sequence has between five and eleven residues, the promoter sequence has between one hundred forty and one hundred seventy residues, the second polyA sequence has between five and eleven residues and the second restriction sequence has between four and seven residues.

14. The composition of matter as recited in claim 5, wherein the composition of matter has a primary structure of SEQ ID NO: 17.

15. A plasmid vector comprising the composition of matter as recited in claim 14.

16. The composition of matter as recited in claim 4, further comprising a first digestion enhancing sequence disposed 5′ and a second digestion enhancing sequence disposed 3′.

17. The composition of matter as recited in claim 16, wherein the composition of matter consists of the first digestion enhancing sequence, the first restriction sequence, the first polyA sequence, the promoter sequence, the nucleotide sequence with a primary structure of SEQ ID NO: 19, the second polyA sequence, the second restriction sequence and the second digestion enhancing sequence.

18. A plasmid vector comprising the composition of matter as recited in claim 17.

19. The composition of matter as recited in claim 16, wherein the first digestion enhancing sequence has between five and one hundred residues, the first restriction sequence has between four and seven residues, the first polyA sequence has between five and eleven residues, the promoter sequence has between one hundred forty and one hundred seventy residues, the second polyA sequence has between five and eleven residues, the second restriction sequence has between four and seven residues and the second digestion enhancing sequence has between five and one hundred residues.

20. A composition of matter comprising a primary structure of SEQ ID NO 18.
